SEQUENCE LISTING (19 sequences)

SEQ ID NO: 1 - DNA, 1086 bp, Aggregatibacter actinomycetemcomitans
aattgttgcgtaaaaggcaattccatatatccgcaaaaaacaagtaccaagcagaccggattaatgctggacatcgcccgacatttttattcacccgaggtgattaaatcctttattgataccatcagcctttccggcggtaattttctgcacctgcatttttccgaccatgaaaactatgcgatagaaagccatttacttaatcaacgtgcggaaaatgccgtgcagggcaaagacggtatttatattaatccttataccggaaagccattcttgagttatcggcaacttgacgatatcaaagcctatgctaaggcaaaaggcattgagttgattcccgaacttgacagcccgaatcacatgacggcgatctttaaactggtgcaaaaagacagaggggtcaagtaccttcaaggattaaaatcacgccaggtagatgatgaaattgatattactaatgctgacagtattacttttatgcaatctttaatgagtgaggttattgatatttttggcgacacgagtcagcattttcatattggtggcgatgaatttggttattctgtggaaagtaatcatgagtttattacgtatgccaataaactatcctactttttagagaaaaaagggttgaaaacccgaatgtggaatgacggattaattaaaaatacttttgagcaaatcaacccgaatattgaaattacttattggagctatgatggcgatacgcaggacaaaaatgaagctgccgagcgccgtgatatgcgggtcagtttgccggagttgctggcgaaaggctttactgtcctgaactataattcctattatctttacattgttccgaaagcttcaccaaccttctcgcaagatgccgcctttgccgccaaagatgttataaaaaattgggatcttggtgtttgggatggacgaaacaccaaaaaccgcgtacaaaatactcatgaaatagccggcgcagcattatcgatctggggagaagatgcaaaagcgctgaaagacgaaacaattcagaaaaacacgaaaagtttattggaagcggtgattcataagacgaatggggatgagtga

SEQ ID NO: 2 - protein, 381 aa, Aggregatibacter actinomycetemcomitans
MNYIKKIILSLFLLGLFSVLNCCVKGNSIYPQKTSTKQTGLMLDIARHFYSPEVIKSFIDTISLSGGNFLHLHFSDHENYAIESHLLNQRAENAVQGKDGIYINPYTGKPFLSYRQLDDIKAYAKAKGIELIPELDSPNHMTAIFKLVQKDRGVKYLQGLKSRQVDDEIDITNADSITFMQSLMSEVIDIFGDTSQHFHIGGDEFGYSVESNHEFITYANKLSYFLEKKGLKTRMWNDGLIKNTFEQINPNIEITYWSYDGDTQDKNEAAERRDMRVSLPELLAKGFTVLNYNSYYLYIVPKASPTFSQDAAFAAKDVIKNWDLGVWDGRNTKNRVQNTHEIAGAALSIWGEDAKALKDETIQKNTKSLLEAVIHKTNGDE

SEQ ID NO: 3 - DNA, 555 bp, Actinobacillus ligniersii
gatcacgagaattatgcattggaaagttcttatttggaacaacgagaagaaaatgccgttgagaaaaacggaacctatttcaatccgaaaacaaataagccgtttctcacttataaacagctcaatgaaattatctattatgccaaagaacgaaatattgaaattgtgcctgaagtcgatagcccgaatcatatgacggcgatttttgatcttttaacccttaagcacggtaaggagtatgtgaaagggctgaaatcgccttatcttgccgaggaaatcgatattaataaccctgaagcggttgaaattatcaaaaccttaatcggtgaagtgatttatatttttgggcattccagccgacactttcatatcggcggagacgaatttagttatgcggtcgaaaacaatcacgaatttattcgttatgtaaatacgctaaatgactttattaataacaaaggactaattacccgtatttggaacgacggtttgattaaaaacaatttaaatgagcttaatcggaatatcgaaattacttattggagctacgacggt

SEQ ID NO: 4 - protein, 185 aa, Actinobacillus ligniersii
DHENYALESSYLEQREENAVEKNGTYFNPKTNKPFLTYKQLNEIIYYAKERNIEIVPEVDSPNHMTAIFDLLTLKHGKEYVKGLKSPYLAEEIDINNPEAVEIIKTLIGEVIYIFGHSSRHFHIGGDEFSYAVENNHEFIRYVNTLNDFINNKGLITRIWNDGLIKNNLNELNRNIEITYWSYDG

SEQ ID NO: 5 - DNA, 558 bp, Aggregatibacter actinomycetemcomitans
gatcatgaaaactatgcgatagaaagccatttacttaatcaacgtgcggaaaatgccgtacagggcaaagacggtatttatattaatccttataccggaaagccattcttgagttatcgacaacttgacgatatcaaagcctatgctaaggcaaaaggcattgagttgattcccgaacttgatagtccgaatcacatgacggcgatctttaaactggtgcaaaaagacagagggatcaagtatcttcaaggattaaaatcacgccaggtagatgatgaaattgatattactaatgctgacagtattgcttttatgcaatctttaatgagtgaggttattgatatttttggcgacacgagtcagcattttcatattggtggcgatgaatttggttattctgtggaaagtaatcatgagtttattacgtatgccaataaactatcctactttttagagaaaaaggggttgaaaacccgaatgtggaatgacggattaattaaaagtacttttgagcaaatcaacccgaatattgaaattacttattggagctatgatggc

SEQ ID NO: 6 - protein, 186 aa, Aggregatibacter actinomycetemcomitans
DHENYAIESHLLNQRAENAVQGKDGIYINPYTGKPFLSYRQLDDIKAYAKAKGIELIPELDSPNHMTAIFKLVQKDRGIKYLQGLKSRQVDDEIDITNADSIAFMQSLMSEVIDIFGDTSQHFHIGGDEFGYSVESNHEFITYANKLSYFLEKKGLKTRMWNDGLIKSTFEQINPNIEITYWSYDG

SEQ ID NO: 7 - DNA, 558 bp, Aggregatibacter aphrophilus
gaccacgaaaattatgctttagaaagcaggttgttgaatcagcgggcagaaaacgcaattttaaataaaaacggaatttatattaatccttacaccaataagcctttcttgagttatcaacagttggatgacattaaagcatatgcaaaattaaaaggtattgagcttattcccgaattagatagcccgaatcacatgacagcgatttttaccttattaaaaaaagaaaaaggaaaaaattatcttcaatcgttaaaatcaccacaaaatgatgaggaaattagcattaccaatccggacagcattgcatttatgcaatccttattaacagaggtaattcatacctttggcgatagcaccaagcattttcatattggcggagatgagtttggttatgatgaaaatagtaatcatgaatttattacctatgccaataaattggctgattttttaagagaaaaaggattaaaaactcgaatttggaatgatggtttaattaaaaataccatagatcaattaaatcctaatattgaaattacctactggagttacgacggc

SEQ ID NO: 8 - protein, 186 aa, Aggregatibacter aphrophilus
DHENYALESRLLNQRAENAILNKNGIYINPYTNKPFLSYQQLDDIKAYAKLKGIELIPELDSPNHMTAIFTLLKKEKGKNYLQSLKSPQNDEEISITNPDSIAFMQSLLTEVIHTFGDSTKHFHIGGDEFGYDENSNHEFITYANKLADFLREKGLKTRIWNDGLIKNTIDQLNPNIEITYWSYDG

SEQ ID NO: 9 - DNA, 1134 bp, Actinobacillus pleuropneumoniae
atgaaaaaagcaattactttatttacattattatgcgcggtacttctctcattcagtaccgcaacttatgcaaacgctatggacttacctaaaaaagaaagcggtctgacgttagatatcgcacgtcgtttctataccgttgatacgataaaacaatttatcgatacgattcatcaggcgggcggcacttttctgcatttacatttttccgatcacgagaattatgcattggaaagttcttatttggaacaacgagaagaaaatgcgaccgagaaaaacggaacctatttcaatccgaaaacaaataagccgtttctcacttataaacagctcaatgaaattatctattatgccaaagaacgaaatattgaaattgtgcctgaagtcgatagcccgaatcatatgacggcgatttttgatcttttaacccttaagcacggaaaggaatacgtaaaagggctaaaatcgccttatatcgccgaggaaatcgatattaataaccccgaagcggttgaagttattaaaaccttaatcggtgaagtgatctatattttcggacattcaagccggcatttccatatcggcggagatgaatttagctatgcggtcgaaaataatcatgaatttattcggtatgtgaataccttaaatgattttatcaattccaaagggctaattacccgtgtttggaatgacggtttgatcaaaaacaacttaagcgaactcaataaaaacattgaaatcacttactggagctacgacggtgacgctcaagccaaagaagatattcaatatcgacgtgaaataagagccgatttgcccgaactgctggcaaacggttttaaggttttaaactataattcttattatttatactttgtgcctaaatccggttctaatattcacaatgacggtaaatatgcggctgaagacgtattaaataactggacattaggtaaatgggacggaaaaaacagctcaaatcacgtacaaaatacgcagaatattatcggttcttctttgtcgatttggggggaacgttccagcgcattaaatgaacaaactattcagcaagcctctaaaaatttattaaaagcggtgatccaaaaaactaatgatccgaaatcgcattag

SEQ ID NO: 10 - protein, 377 aa, Actinobacillus pleuropneumoniae
MKKAITLFTLLCAVLLSFSTATYANAMDLPKKESGLTLDIARRFYTVDTIKQFIDTIHQAGGTFLHLHFSDHENYALESSYLEQREENATEKNGTYFNPKTNKPFLTYKQLNEIIYYAKERNIEIVPEVDSPNHMTAIFDLLTLKHGKEYVKGLKSPYIAEEIDINNPEAVEVIKTLIGEVIYIFGHSSRHFHIGGDEFSYAVENNHEFIRYVNTLNDFINSKGLITRVWNDGLIKNNLSELNKNIEITYWSYDGDAQAKEDIQYRREIRADLPELLANGFKVLNYNSYYLYFVPKSGSNIHNDGKYAAEDVLNNWTLGKWDGKNSSNHVQNTQNIIGSSLSIWGERSSALNEQTIQQASKNLLKAVIQKTNDPKSH

SEQ ID NO: 11 - DNA, 1212 bp, Aggregatibacter aphrophilus
gtgttttattgcattctttccttttttgaggcttttatgaaaaatataaaaaaaactatctttttacttccatctttgcttttgttgaacccgttaaatagtcttgccgatgtaaagatgtcttctgataattccaatataaattcgccaacagtgcaaaaaacaactttaaaggaaagcggattaatgctggatattgcaaggcacttttatccgcctgaagttattaaatcttttattgatactattagccagtcgggcgggacatttctgcatttacatttttccgatcacgaaaattatgctttagaaagtcggttgctgaatcagcgtgtggaaaatgcgattcaagataaaagcggaatttatattaatccttacaccaataaacccttcttgagctatcaacagcttgatgacattaaagcatatgcaaaatcaaaaggcattgagcttattcccgaactggatagcccgaatcacatgacggcgatttttacgttattaaaacaagaaaagggggaagcctatcttcaatctttaaaatcaccacaaaatgatgacgaaattagcattaccaatccggacagtattgagtttatgcaatctttgttggctgaagttattcatatttttggtgacagtactaagcattttcatattggtggtgatgagtttggttatgatgaaaatagtaatcatgaatttattgcctatgccaataaattagctgattttttaagagaaaaaggattaaaaacccgaatttggaatgatggtttaattaaaaataccgtcgatcaattaaatcctaatattgaaattacctactggagttatgatggtgatacggaagatgaaggcgaggcaaatcgacgtcgtcatctgcgggtgagtctgccggaattagtcgaaaaaggttttactgttttaaattataattcttattatctttatataaatcctaaaacctcttcaacgctttcgcatgatgcgagctttgctcaaaaagacgtcttaaatcattgggatcttagtgtttgggatggacaaaatacggaaaataaagcacaagatacccataaaattgccggcgcggctttgtctatttggggagaagatgtcaaggccttgaaaagtgaagccattcagaaaaatacaaaaggcttactggaggcggtgattcaaaagacgaacagtgagattaaataa

SEQ ID NO: 12 - protein, 403 aa, Aggregatibacter aphrophilus
MFYCILSFFEAFMKNIKKTIFLLPSLLLLNPLNSLADVKMSSDNSNINSPTVQKTTLKESGLMLDIARHFYPPEVIKSFIDTISQSGGTFLHLHFSDHENYALESRLLNQRVENAIQDKSGIYINPYTNKPFLSYQQLDDIKAYAKSKGIELIPELDSPNHMTAIFTLLKQEKGEAYLQSLKSPQNDDEISITNPDSIEFMQSLLAEVIHIFGDSTKHFHIGGDEFGYDENSNHEFIAYANKLADFLREKGLKTRIWNDGLIKNTVDQLNPNIEITYWSYDGDTEDEGEANRRRHLRVSLPELVEKGFTVLNYNSYYLYINPKTSSTLSHDASFAQKDVLNHWDLSVWDGQNTENKAQDTHKIAGAALSIWGEDVKALKSEAIQKNTKGLLEAVIQKTNSEIK

SEQ ID NO: 13 - DNA, 1026 bp, Aggregatibacter aphrophilus
atgctggatattgcaaggcacttttatccgcctgaagttattaaatcttttattgatactattagccagtcgggcggaacatttctgcatttacatttttccgatcacgaaaattatgctttagaaagcaggttgttgaatcagcgggcagaaaacgcaattttaaataaaaacggaatttatattaatccttacaccaataagcctttcttgagttatcaacagttggatgacattaaagcatatgcaaaattaaaaggtgttgagcttattcccgaattagatagcccgaatcacatgacagcgatttttaccttattaaaaaaagaaaaaggaaaaaattatcttcaatcgttaaaatcaccacaaaatgatgaggaaattagcattaccaatccggacagcattgcatttatgcaatccttattaactgaggtaattcatacctttggcgatagcaccaagcattttcatattggcggagatgagtttggttatgatgaaaatagtaatcatgaatttattgcctatgccaataaattagctgattttttaagagaaaaaggattaaaaacccgaatttggaatgatggtttaattaaaaataccgtcgatcaattaaatcctaatattgaaattacctactggagttatgacggtgatacggaagatgaaggcgaggcaaatagacgtcgtcatcttcgggtgagtttgccggaattggtcgaaaaaggttttactgttttaaattataattcttattatctttatataaatcctaaaacctcttcaacgctttctcatgatgcgagctttgctcaaaaagacgtcttaaatcattgggatcttagtgtttgggatggacaaaatatggaaaataaagcacaagatacccataaaattgccggcgcggctttgtctatttggggggaagatgtcaaggccttgaaaagtgaagccattcagaaaaatacaaaaggcttactggaggcggtgattaaaaagacaaatagtgagattaaataa

SEQ ID NO: 14 - protein, 341 aa, Aggregatibacter aphrophilus
MLDIARHFYPPEVIKSFIDTISQSGGTFLHLHFSDHENYALESRLLNQRAENAILNKNGIYINPYTNKPFLSYQQLDDIKAYAKLKGVELIPELDSPNHMTAIFTLLKKEKGKNYLQSLKSPQNDEEISITNPDSIAFMQSLLTEVIHTFGDSTKHFHIGGDEFGYDENSNHEFIAYANKLADFLREKGLKTRIWNDGLIKNTVDQLNPNIEITYWSYDGDTEDEGEANRRRHLRVSLPELVEKGFTVLNYNSYYLYINPKTSSTLSHDASFAQKDVLNHWDLSVWDGQNMENKAQDTHKIAGAALSIWGEDVKALKSEAIQKNTKGLLEAVIKKTNSEIK

SEQ ID NO: 15 - DNA, 30 bp, Artificial Sequence
  Description of Artificial Sequence Synthetic primer
caccatgaattgttgcgtaaaaggcaattc

SEQ ID NO: 16 - DNA, 4 bp, Artificial Sequence
  Description of Artificial Sequence Synthetic oligonucleotide
cacc

SEQ ID NO: 17 - DNA, 42 bp, Artificial Sequence
  Description of Artificial Sequence Synthetic primer
ttagtggtggtggtggtggtgctcatccccattcgtcttatg

SEQ ID NO: 18 - DNA, 1107 bp, Artificial Sequence
  Description of Artificial Sequence Synthetic polynucleotide
atgaattgttgcgtaaaaggcaattccatatatccgcaaaaaacaagtaccaagcagaccggattaatgctggacatcgcccgacatttttattcacccgaggtgattaaatcctttattgataccatcagcctttccggcggtaattttctgcacctgcatttttccgaccatgaaaactatgcgatagaaagccatttacttaatcaacgtgcggaaaatgccgtgcagggcaaagacggtatttatattaatccttataccggaaagccattcttgagttatcggcaacttgacgatatcaaagcctatgctaaggcaaaaggcattgagttgattcccgaacttgacagcccgaatcacatgacggcgatctttaaactggtgcaaaaagacagaggggtcaagtaccttcaaggattaaaatcacgccaggtagatgatgaaattgatattactaatgctgacagtattacttttatgcaatctttaatgagtgaggttattgatatttttggcgacacgagtcagcattttcatattggtggcgatgaatttggttattctgtggaaagtaatcatgagtttattacgtatgccaataaactatcctactttttagagaaaaaagggttgaaaacccgaatgtggaatgacggattaattaaaaatacttttgagcaaatcaacccgaatattgaaattacttattggagctatgatggcgatacgcaggacaaaaatgaagctgccgagcgccgtgatatgcgggtcagtttgccggagttgctggcgaaaggctttactgtcctgaactataattcctattatctttacattgttccgaaagcttcaccaaccttctcgcaagatgccgcctttgccgccaaagatgttataaaaaattgggatcttggtgtttgggatggacgaaacaccaaaaaccgcgtacaaaatactcatgaaatagccggcgcagcattatcgatctggggagaagatgcaaaagcgctgaaagacgaaacaattcagaaaaacacgaaaagtttattggaagcggtgattcataagacgaatggggatgagcaccaccaccaccaccactaa

SEQ ID NO: 19 - protein, 368 aa, Artificial Sequence
  Description of Artificial Sequence Synthetic polypeptide
MNCCVKGNSIYPQKTSTKQTGLMLDIARHFYSPEVIKSFIDTISLSGGNFLHLHFSDHENYAIESHLLNQRAENAVQGKDGIYINPYTGKPFLSYRQLDDIKAYAKAKGIELIPELDSPNHMTAIFKLVQKDRGVKYLQGLKSRQVDDEIDITNADSITFMQSLMSEVIDIFGDTSQHFHIGGDEFGYSVESNHEFITYANKLSYFLEKKGLKTRMWNDGLIKNTFEQINPNIEITYWSYDGDTQDKNEAAERRDMRVSLPELLAKGFTVLNYNSYYLYIVPKASPTFSQDAAFAAKDVIKNWDLGVWDGRNTKNRVQNTHEIAGAALSIWGEDAKALKDETIQKNTKSLLEAVIHKTNGDEHHHHHH

(19) United States
(12) Patent Application Publication
(10) Pub. No.: US 2014/0020138 A1
(43) Pub. Date: Jan. 16, 2014

(54) TRANSGENIC PLANTS EXPRESSING DISPERSINB

(71) Applicants: Chandran Ragunath, Malden, MA (US); Mayilvahanan Shanmugam, Huntington, NY (US); Jeffrey B. Kaplan, Monsey, NY (US); Narayanan Ramasubbu, East Windsor, NJ (US)

(72) Inventors: Chandran Ragunath, Malden, MA (US); Mayilvahanan Shanmugam, Huntington, NY (US); Jeffrey B. Kaplan, Monsey, NY (US); Narayanan Ramasubbu, East Windsor, NJ (US)

(73) Assignee: UNIVERSITY OF MEDICINE AND DENTISTRY OF NEW JERSEY, New Brunswick, NJ (US)

(21) Appl. No.: 14/006,695

(22) PCT Filed: Mar. 23, 2012
(86) PCT No.: PCT/US12/30390

§ 371 Date: Sep. 23, 2013

Related U.S. Application Data
(60) Provisional application No. 61/466,625, filed on Mar. 23, 2011.

(51) Int. Cl. C12N 15/82

(52) U.S. Cl. CPC C12N 15/8281
USPC 800/301

(57) ABSTRACT

Methods and compositions for producing genetically transformed plants exhibiting antibiofilm activity against bacterial pathogens are disclosed. Bacterial cells and plant transformation vectors include dspB genes encoding DispersinB antibiofilm enzymes of Aggregatibacter actinomycetemcomitans.

### PRIORITY CLAIM

This patent application is a U.S. National Phase of International Patent Application No. PCT/US2012/030390, filed Mar. 23, 2012, which claims priority to U.S. Provisional Patent Application No. 61/466,625, filed Mar. 23, 2011, the disclosures of which are incorporated herein by reference in their entirety.

### BACKGROUND

Infectious plant diseases cause human suffering by reducing food supplies and result in enormous economic losses. One-third of the total crop loss in the world can be attributed directly to plant disease. In the U.S. alone, crop losses to plant pathogens total approximately $33 billion per year. Although pesticides have successfully controlled disease, their continued and increasing use poses a risk to our health and the environment. Alternatively, use of high yield crop varieties can also improve productivity, but the use of genetically uniform varieties for cultivation over enormous areas is susceptible to devastating epidemics. Thoughtful application of the plant's own defense mechanisms combined with understanding of the complex ecology of real-world disease processes has the potential for more effective protection against plant pathogens.

Soft rot Pectobacteriam carotovorum subspecies such as P. carotovora subsp. carotovora (formerly Erwinia carotovora, also known as Pcc) is economically important, because it causes serious damage worldwide on a wide variety of plants. For instance, reduction in the yield of potato from this pathogen can be as high as 24% of the worldwide production. These species cause diseases mainly by secretion of cell-wall-degrading enzymes such as pectate lyase (Pel), polygalacturonase (Peh), cellulase (Cel) and protease (Prt). In addition, secondary factors such as exopolysaccharides (EPS), lipopolysaccharide (LPS), iron assimilation, the Hrp system and motility also contribute to virulence in soft-rot Erwinia spp.

Other plant diseases such as citrus canker in citrus fruits, pith necrosis in tomato, red-xylem disease, pink eye and bruise infection of potato, bacterial wilt in potato, tobacco, tomato and groundnut are caused by bacterial pathogens. Citrus canker is caused by Xanthomonas axonopodis and shows erumpent and corky lesions on all aerial parts of mature citrus trees such as leaves, stems, and fruits. Bacterial wilt caused by Ralstonia solanacearum was reported for the first time at the end of the 19^{th} century on potato, tobacco, tomato and groundnut in Asia, southern USA and South America. Pseudomonas fluorescens causes red-xylem disease, pink-eye disease, and bruise cracks in potato tubers. P. fluorescens causes pith necrosis in tomato. The symptoms include yellowing and wilting of lower leaves which progresses upwards, brown areas on stems.

DispersinB is an enzyme that can be produced by the gram-negative oral bacterium Aggregatibacter actinomycetemcomitans. DispersinB cleaves the N-acetyl-D-glucosamine residues in β(1,6) linkage, which disrupts bacterial biofilm formation and/or growth. Studies have shown DispersinB is effective in inhibiting the formation and growth of oral bacterial biofilm in vitro and on indwelling medical devices in vivo. However, DispersinB is not surface bound or secreted when expressed in plants. The DispersinB polypeptide remains localized in the cytosol.

### SUMMARY

Plant crops that are susceptible to bacterial diseases, such as soft rot, bacterial wilt, red xylem and pith necrosis, are targets to introduce DispersinB into crops to inhibit biofilm formation and growth. Because bacteria in biofilms can be 1000 times more resistant to antimicrobials, transgenic plants expressing DispersinB can potentially improve the efficacy of current antimicrobial treatments in crops.

A transformed plant, plant cell, plant tissue, and plant seed with a DispersinB polynucleotide are described herein. Transformed plants, plant cells, plant tissue, and plant seeds able to express a DispersinB polypeptide inhibit biofilm formation and growth. A DispersinB enzyme can be a polypeptide of SEQ ID NO:2 or any ortholog thereof that cleaves the N-acetyl-D-glucosamine residues in β(1,6) linkage. Another embodiment includes a transgenic plant expressing a fusion protein, wherein the fusion protein is a DispersinB polypeptide, and a second polypeptide. An embodiment includes a transgenic plant comprising a DispersinB polynucleotide. A transgenic plant may also include a variant polynucleotide or polynucleotide fragment of a DispersinB enzyme, wherein the variant or fragment encodes a polypeptide that can cleave a N-acetyl-D-glucosamine residue in β(1,6) linkage. Further, a vector, such as an expression vector, includes a DispersinB polynucleotide. A DispersinB polynucleotide encodes the A. actinomycetemcomitans DispersinB polynucleotide (SEQ ID NO:2), orthologs thereof such as SEQ ID NOs: 4, 6, 8, and 10, and active fragments thereof. A DispersinB polynucleotide is operably linked to one or more control sequences, such as a promoter, an enhancer, or a ribosome binding site. Suitable promoters include inducible promoters.

A transgenic plant that includes a polynucleotide encoding a fusion protein is described wherein the fusion protein includes a DispersinB or active fragment thereof linked to a second polypeptide. A suitable second polypeptide is an antimicrobial polypeptide. A transgenic plant may also include a variant polynucleotide or polynucleotide fragment of a DispersinB enzyme linked to a second polynucleotide, wherein the variant or fragment of a DispersinB enzyme encodes a polypeptide that can cleave a N-acetyl-D-glucosamine residue in β(1,6) linkage. Further, a vector, such as an expression vector, includes a DispersinB polynucleotide linked to a second polynucleotide that encodes a second polypeptide. A DispersinB polynucleotide includes a polynucleotide that encodes the A. actinomycetemcomitans DispersinB polynucleotide (SEQ ID NO:2), orthologs thereof such as SEQ ID NOs: 4, 6, 8, and 10. A DispersinB polynucleotide is operably linked to one or more control sequences, such as a promoter, an enhancer, or a ribosome binding site. A suitable promoter is an inducible promoter. A promoter may also be a tissue specific promoter such as a root specific or fruit specific promoter.

Methods of expressing a DispersinB polypeptide in a plant are disclosed. One method includes using a plant expressing a DispersinB polypeptide, variant, or fragment thereof, wherein the transgenic plant is resistant to bacterial infection. Another embodiment includes a plant expressing a DispersinB polypeptide, variant, or fragment thereof, wherein the transgenic plant inhibits microbial biofilm formation and/or growth. Another embodiment includes expressing a DispersinB polypeptide, variant, or fragment thereof, wherein the transgenic plant inhibits microbial biofilm formation and/or growth in combination with an application of an antimicrobial. The aforementioned methods can also include substituting a fusion protein for the DispersinB polypeptide, wherein the fusion protein is a DispersinB polypeptide, variant, or fragment thereof linked to a second polypeptide.

A DispersinB polypeptide expressed in a plant may be extracted. The extract include a crude extract or it can be a substantially purified extract, e.g. between 95 and 100% pure. A composition includes a plant extract of DispersinB in combination with at least one antimicrobial, wherein the extract includes a crude extract or a substantially pure extract that is less than 100% pure, but pure enough to be effective.

### BRIEF DESCRIPTION OF THE FIGURES

FIG. 1 shows a plasmid map for plant transformation cassette vector pMDC32-DspB.

FIG. 2 shows that DspB-expressing-transgenic tobacco plants produced substantial amount of the biologically active enzyme, whereas there was no biological activity in the vector-transformed control plants.

FIG. 3 shows that the biologically active DispersinB enzyme from the transgenic plants not only detaches the preformed Staphylococcus epidermidis biofilm but also inhibits the biofilm formation.

FIG. 4 shows (a) results of Staphylococcus epidermidis biofilm detachment assay in 96-well polystyrene microtiter plates; (b) Residual biofilms in wells (4a) after treatment, as measured by absorbance at 590 nm. Results based on triplicate measurements with standard deviations shown as error bars).

FIG. 5 (a) shows inhibition of Staphylococcus epidermidis biofilm formation by transgenic tobacco leaf extract; Bottom panel shows the detachment by DspB at varying concentrations. (b) Residual biofilms in wells (5a) after treatment, as measured by absorbance at 590 nm.

FIG. 6 shows the results of western blot providing the direct evidence of the successful expression of integrated DspB gene into the plant genome.

FIG. 7 shows (a) Bacterial extracts from 24-h-old cultures of Pectobacterium carotovorum subsp. carotovorum cells immunoblotted with antibody raised against deacetylated PNAG; (b) Lane A, polysaccharide from P. carotovorum; lane B, DspB-treated polysaccharide from P. carotovorum; lane C, N-acetylglucosamine; lane D, tetramer of β(1,6)linked N-acetylglucosamine; and lane E, chitin tetrasaccharide. (b) Inhibition of P. carotovorum biofilm formation by DspB. (c) Pectobacterium carotovorum biofilm detachment by DspB.

FIG. 8 show tobacco leaf infected with P. carotovorum. Left panel, no DspB was applied topically. Right panel, DspB was applied topically. Both treatment show typical maceration suggesting that topical application of DspB does not prevent or inhibit P. carotovorum infection.

FIG. 9 shows evidence for the resistance of the DispersinB expressing transformed plants (D10, D12, D17, D21, and D23) to relatively a high inoculum of plant pathogen P. carotovorum in contrast to control plants (C1) displaying typical maceration symptoms within 24 h after inoculation.

FIG. 10 shows the viability of P. carotovorum from infected sites (the bottom spots) of a control leaf (C1) and the transgenic leaves in FIG. 8. Pcc were collected from leaves C1, D17, and D21 in FIG. 8 and used to infect control plant leaves; All three control leaves show maceration from the bacterial inoculum.

### DETAILED DESCRIPTION OF THE INVENTION

### Definitions

The term “active fragment” refers to smaller portions of the DspB polypeptide that retains the ability to disperse bacteria or fungi.

The term “antimicrobial” means a compound or a composition that kills or slows/stops the growth of microorganisms, including, but not limited to bacteria and yeasts.

The term “biofilm embedded microorganisms” refers to any microorganism that forms a biofilm during colonization and proliferation on a surface, including, but not limited to, gram-positive bacteria (e.g., Staphylococcus epidermidis), gram-negative bacteria (e.g., Pseudomonas aeruginosa), and/or fungi (e.g., Candida albicans).

The term “biofilm formation” means the attachment of microorganisms to surfaces and subsequent development of multiple layers of cells.

The term “disperse” refers to individual bacterial or fungal cells detaching from a surface or detaching from a biofilm. The term “disperse” also refers to disaggregation of autoaggregating bacterial or fungal biofilm cells.

The term “inhibition” or “inhibiting” refers to a decrease of biofilm associated microorganism formation and/or growth. The microorganisms includes bacteria (e.g., streptococci) and fungi (e.g., Candida spp.)

In general, the “wild type” sequence for a given protein is the sequence that is most common in nature. Similarly, a “wild type” gene sequence is the sequence for that gene which is most commonly found in nature. Mutations may be introduced into a “wild type” gene (and thus the protein it encodes) either through natural processes or through man induced means. The products of such processes are “variant” or “mutant” forms of the original “wild type” protein or gene.

A “variant” of a polypeptide refers to a polypeptide that contains an amino acid sequence that differs from a wild type or reference sequence. A variant polypeptide can differ from a wild type or reference sequence due to a deletion, insertion, or substitution of a nucleotide(s) relative to said reference or wild type nucleotide sequence. The reference or wild type sequence can be a full-length native polypeptide sequence or any other fragment of a full-length polypeptide sequence. A polypeptide variant generally has at least about 80% amino acid sequence identity with the reference sequence, but may include at least 85% amino acid sequence identity with the reference sequence, 86% amino acid sequence identity with the reference sequence, 87% amino acid sequence identity with the reference sequence, 88% amino acid sequence identity with the reference sequence, 89% amino acid sequence identity with the reference sequence, 90% amino acid sequence identity with the reference sequence, 91% amino acid sequence identity with the reference sequence, 92% amino acid sequence identity with the reference sequence, 93% amino acid sequence identity with the reference sequence, 94% amino acid sequence identity with the reference sequence, 95% amino acid sequence identity with the reference sequence, 96% amino acid sequence identity with the reference sequence, 97% amino acid sequence identity with the reference sequence, 98% amino acid sequence identity with the reference sequence, or 99% amino acid sequence identity with the reference sequence.

“Percent (%) nucleic acid sequence identity” is defined as the percentage of nucleotides in a candidate sequence that are identical with the nucleotides in a reference polypeptide-encoding nucleic acid sequence, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For purposes herein, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:

100 times the fraction W/Z,

where W is the number of nucleotides scored as identical matches by the sequence alignment program in that program's alignment of C and D, and where Z is the total number of nucleotides in D. Where the length of nucleic acid sequence C is not equal to the length of nucleic acid sequence D, the % nucleic acid sequence identity of C to D will not equal the % nucleic acid sequence identity of D to C.

The terms “nucleic acid,” “nucleotide,” “polynucleotide,” and “oligonucleotide” are used interchangeably. These terms refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The polynucleotide can be single stranded or double stranded.

The term “protein” has an amino acid sequence that is longer than a peptide. A “peptide” contains 2 to about 50 amino acid residues. The term “polypeptide” includes proteins and peptides. Examples of proteins include, but are not limited to, antibodies, enzymes, lectins and receptors; lipoproteins and lipopolypeptides; and glycoproteins and glycopolypeptides.

The term “expression” refers to the process by which a polynucleotide is transcribed into mRNA and/or the process by which the transcribed mRNA (also referred to as “transcript”) is subsequently translated into peptides, polypeptides, or proteins.

The term “overexpressed” or “overexpression” as applied to nucleotide sequence or polypeptide sequence refers to expression of the sequence in greater quantity when compared to that detected in a control.

A nucleic acid sequence or polynucleotide is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading frame. Linking can be accomplished by ligation at convenient restriction sites. If such sites do not exist, synthetic oligonucleotide adaptors or linkers are used in accordance with conventional practice.

The term “control sequences” refer to DNA sequences necessary for the expression of an operably linked coding sequence in a particular host organism. The control sequences that are suitable for prokaryotes, for example, include a promoter, optionally an operator sequence, a ribosome binding site, and possibly, other as yet poorly understood sequences. Eukaryotic cells are known to utilize promoters, polyadenylation signals, and enhancers.

“Plasmids” are designated by a lower case “p” preceded and/or followed by capital letters and/or numbers. The starting plasmids herein are either commercially available, publicly available on an unrestricted basis, or can be constructed from available plasmids in accord with published procedures. In addition, equivalent plasmids to those described are known in the art and will be apparent to the ordinarily skilled artisan.

As used herein, the phrase “induce expression” means to increase the amount or rate of transcription and/or translation from specific genes by exposure of the cells containing such genes to an effector or inducer reagent or condition.

An “inducer” is a chemical or physical agent which, when applied to a population of cells, will increase the amount of transcription from specific genes. These are usually small molecules whose effects are specific to particular operons or groups of genes, and can include sugars, phosphate, alcohol, metal ions, hormones, heat, cold, and the like. For example, isopropyl (beta)-D-thiogalactopyranoside (IPTG) and lactose are inducers of the tacII promoter, and L-arabinose is a suitable inducer of the arabinose promoter.

A “fusion protein” and a “fusion polypeptide” refer to a polypeptide having two portions covalently linked together, where each of the portions is a polypeptide having a different property. The property may be a biological property, such as activity in vitro or in vivo. The property may also be a simple chemical or physical property, such as binding to a target antigen, catalysis of a reaction, etc. The two portions may be linked directly by a single peptide bond or through a peptide linker containing one or more amino acid residues. Generally, the two portions and the linker will be in reading frame with each other. Preferably, the two portions of the polypeptide are obtained from heterologous or different polypeptides.

“PCR” refers to the technique in which minute amounts of a specific piece of nucleic acid, RNA and/or DNA, are amplified as described in U.S. Pat. No. 4,683,195. PCR can be used to amplify specific RNA sequences, specific DNA sequences from total genomic DNA, and cDNA transcribed from total cellular RNA, bacteriophage or plasmid sequences, etc.

The term “primer” refers to an oligonucleotide capable of acting as a point of initiation of synthesis along a complementary strand when conditions are suitable for synthesis of a primer extension product. The synthesizing conditions include the presence of four different deoxyribonucleotide triphosphates and at least one polymerization-inducing agent such as reverse transcriptase or DNA polymerase. These are present in a suitable buffer, which may include constituents which are co-factors or which affect conditions such as pH and the like at various suitable temperatures. A primer is preferably a single strand sequence, such that amplification efficiency is optimized, but double stranded sequences can be utilized.

The term “substantially purified”, as used herein, refers to a molecule separated from substantially all other molecules normally associated with it in its native state. More preferably, a substantially purified molecule is the predominant species present in a preparation. A substantially purified molecule may be greater than 60%, 65%, 70%, 75%, 80%, 85%, 90%, 91%, 92%, 93%, 94%, 95%, 96%, 97%, 98%, 99%, or 100% free from other molecules present in the natural mixture. The term “substantially purified” is not intended to encompass molecules present in their native state.

The term “plant” refers to whole plants, plant organs (e.g., leaves, stems, roots, etc.), seeds, plant cells, propagules, embryos and progeny of the same. Plant cells can be differentiated or undifferentiated (e.g. callus, suspension culture cells, protoplasts, leaf cells, root cells, phloem cells, and pollen). “Transgenic plants” or “transformed plants” or “stably transformed” plants, cells or tissues refer to plants that have incorporated or integrated exogenous nucleic acid sequences or DNA fragments into the plant cell.

The term “stable transformation” refers to introduction of the nucleotide construct into a plant, integrates into the genome of the plant, and is capable of being inherited by progeny thereof.

### DispersinB (DspB)

Biofilm-embedded Aggregatibacter (formerly Actinobacillus) actinomycetemcomitans can release individual cells into liquid medium. These detached cells can attach to the surface of a culture apparatus and start a new colony. The dspB gene encodes a 381 amino acid soluble β-N-acetylglucosaminidase that is responsible for the detachment/dispersion of A. actinomycetemcomitans. This polypeptide is referred to as DispersinB (DspB). The first 20 amino acids are a signal peptide, and amino acids 21-381 are the mature polypeptide. The mature DspB polypeptide has the following sequence (amino acids 21-381 of SEQ ID NO:2; Accession No. AAP31025.1 GI:30420960):

| | |
|---|---|
| 1 | nccvkgnsiy pqktstkqtg lmldiarhfy speviksfid tislsggnfl hlhfsdheny |
| 61 | aieshllnqr aenavqgkdg iyinpytgkp flsyrqlddi kayakakgie lipeldspnh |
| 121 | mtaifklvqk drgvkylqgl ksrqvddeid itnadsitfm qslmsevidi fgdtsqhfhi |
| 181 | ggdefgysve snhefityan klsyflekkg lktrmwndgl ikntfeqinp nieitywsyd |
| 241 | gdtqdkneaa errdmrvslp ellakgftvl nynsyylyiv pkasptfsqd aafaakdvik |
| 301 | nwdlgvwdgr ntknrvqnth eiagaalsiw gedakalkde tiqkntksll eavihktngd |
| 361 | e |

Orthologs of DispersinB polypeptide have been identified and are depicted in SEQ ID Nos: 2, 4, 6, 8, 10, 12, and 14 respectively (see Table 2). For instance, the closely related Actinobacillus pleuropneumoniae also encodes a DspB, a 377 amino acid polypeptide that includes a signal peptide from amino acids 1 to 34. The A. pleuropneumoniae DspB has a full-length polypeptide sequence of SEQ ID NO:10 (Accession No. AAT46094.1 GI:48727581). Further, Aggregatibacter aphrophilus (formerly Haemophilus aphrophilus) also contains an ortholog DspB, which is a 341 amino acid polypeptide (SEQ ID NO:8) that includes a signal peptide from amino acids 1 to 34.

Embodiments of the invention also include active fragments and variants of SEQ ID Nos: 2, 4, 6, 10, 12, and 14. DspB active fragments and variants only include those fragments and variants that retain an ability to disperse bacterial or fungal cells from the biofilm.

A substrate for DspB is a high-molecular weight hexosamine-containing extracellular polysaccharide Adhesion encoded in the pgaABCD locus and pgaCD in A. acetinomycetemcomitans and A. pleuropneumoniae, respectively (Kaplan et al., 2004, J. Bacteriol. 186:8213-8220). These polysaccharide adhesins are a component of the Aggregatibacter biofilm and act as a protective barrier for cells of a biofilm. Aggregatibacter PGA is structurally and functionally similar to E. coli PGA and S. epidermidis PIA, both polysaccharides comprising N-acetyl-D-glucosamine residues in β(1,6) linkage (Kaplan et al., 2004). Methods and compositions disclosed herein are used to detach bacterial cells other than A. acetinomycetemcomitans, A. pleuropneumoniae, A. ligniersii, or A. aphrophilus.

TABLE 1
DspB Nucleotide Sequences

| SEQ | | |
|---|---|---|
| ID NO: | Origin | Sequence |
| 1 | Aggregatibacter | AATTGTTGCGTAAAAGGCAATTCCATATATCCGCAAAAAACAAGTACCAA |
| | actinomycetemcomitans | GCAGACCGGATTAATGCTGGACATCGCCCGACATTTTTATTCACCCGAGG |
| | strain CU1000N | TGATTAAATCCTTTATTGATACCATCAGCCTTTCCGGCGGTAATTTTCTG |
| | | CACCTGCATTTTTCCGACCATGAAAACTATGCGATAGAAAGCCATTTACT |
| | | TAATCAACGTGCGGAAAATGCCGTGCAGGGCAAAGACGGTATTTATATTA |
| | | ATCCTTATACCGGAAAGCCATTCTTGAGTTATCGGCAACTTGACGATATC |
| | | AAAGCCTATGCTAAGGCAAAAGGCATTGAGTTGATTCCCGAACTTGACAG |
| | | CCCGAATCACATGACGGCGATCTTTAAACTGGTGCAAAAAGACAGAGGGG |
| | | TCAAGTACCTTCAAGGATTAAAATCACGCCAGGTAGATGATGAAATTGAT |
| | | ATTACTAATGCTGACAGTATTACTTTTATGCAATCTTTAATGAGTGAGGT |
| | | TATTGATATTTTTGGCGACACGAGTCAGCATTTTCATATTGGTGGCGATG |
| | | AATTTGGTTATTCTGTGGAAAGTAATCATGAGTTTATTACGTATGCCAAT |
| | | AAACTATCCTACTTTTTAGAGAAAAAAGGGTTGAAAACCCGAATGTGGAA |
| | | TGACGGATTAATTAAAAATACTTTTGAGCAAATCAACCCGAATATTGAAA |
| | | TTACTTATTGGAGCTATGATGGCGATACGCAGGACAAAAATGAAGCTGCC |
| | | GAGCGCCGTGATATGCGGGTCAGTTTGCCGGAGTTGCTGGCGAAAGGCTT |
| | | TACTGTCCTGAACTATAATTCCTATTATCTTTACATTGTTCCGAAAGCTT |
| | | CACCAACCTTCTCGCAAGATGCCGCCTTTGCCGCCAAAGATGTTATAAAA |
| | | AATTGGGATCTTGGTGTTTGGGATGGACGAAACACCAAAAACCGCGTACA |
| | | AAATACTCATGAAATAGCCGGCGCAGCATTATCGATCTGGGGAGAAGATG |
| | | CAAAAGCGCTGAAAGACGAAACAATTCAGAAAAACACGAAAAGTTTATTG |
| | | GAAGCGGTGATTCATAAGACGAATGGGGATGAGTGA |
| | | (Accession No. AY228551.1; GI: 30420959) |
| 3 | Actinobacillus ligniersii | GATCACGAGAATTATGCATTGGAAAGTTCTTATTTGGAACAACGAGA |
| | strain 19393 | AGAAAATGCCGTTGAGAAAAACGGAACCTATTTCAATCCGAAAACAA |
| | | ATAAGCCGTTTCTCACTTATAAACAGCTCAATGAAATTATCTATTAT |
| | | GCCAAAGAACGAAATATTGAAATTGTGCCTGAAGTCGATAGCCCGAA |
| | | TCATATGACGGCGATTTTTGATCTTTTAACCCTTAAGCACGGTAAGG |
| | | AGTATGTGAAAGGGCTGAAATCGCCTTATCTTGCCGAGGAAATCGAT |
| | | ATTAATAACCCTGAAGCGGTTGAAATTATCAAAACCTTAATCGGTGA |
| | | AGTGATTTATATTTTTGGGCATTCCAGCCGACACTTTCATATCGGCG |
| | | GAGACGAATTTAGTTATGCGGTCGAAAACAATCACGAATTTATTCGT |
| | | TATGTAAATACGCTAAATGACTTTATTAATAACAAAGGACTAATTAC |
| | | CCGTATTTGGAACGACGGTTTGATTAAAAACAATTTAAATGAGCTTA |
| | | ATCGGAATATCGAAATTACTTATTGGAGCTACGACGGT |
| | | (DD357870.1 GI: 118145582) |
| 5 | Aggregatibacter | GATCATGAAAACTATGCGATAGAAAGCCATTTACTTAATCAACGTGC |
| | actinomycetemcomitans | GGAAAATGCCGTACAGGGCAAAGACGGTATTTATATTAATCCTTATA |
| | strain IDH781 | CCGGAAAGCCATTCTTGAGTTATCGACAACTTGACGATATCAAAGCC |
| | | TATGCTAAGGCAAAAGGCATTGAGTTGATTCCCGAACTTGATAGTCC |
| | | GAATCACATGACGGCGATCTTTAAACTGGTGCAAAAAGACAGAGGGA |
| | | TCAAGTATCTTCAAGGATTAAAATCACGCCAGGTAGATGATGAAATT |
| | | GATATTACTAATGCTGACAGTATTGCTTTTATGCAATCTTTAATGAG |
| | | TGAGGTTATTGATATTTTTGGCGACACGAGTCAGCATTTTCATATTG |
| | | GTGGCGATGAATTTGGTTATTCTGTGGAAAGTAATCATGAGTTTATT |
| | | ACGTATGCCAATAAACTATCCTACTTTTTAGAGAAAAAGGGGTTGAA |
| | | AACCCGAATGTGGAATGACGGATTAATTAAAAGTACTTTTGAGCAAA |
| | | TCAACCCGAATATTGAAATTACTTATTGGAGCTATGATGGC |
| 7 | Aggregatibacter | GACCACGAAAATTATGCTTTAGAAAGCAGGTTGTTGAATCAGCGGGC |
| | aphrophilus | AGAAAACGCAATTTTAAATAAAAACGGAATTTATATTAATCCTTACA |
| | strain NJ8700 | CCAATAAGCCTTTCTTGAGTTATCAACAGTTGGATGACATTAAAGCA |
| | | TATGCAAAATTAAAAGGTATTGAGCTTATTCCCGAATTAGATAGCCC |
| | | GAATCACATGACAGCGATTTTTACCTTATTAAAAAAAGAAAAAGGAA |
| | | AAAATTATCTTCAATCGTTAAAATCACCACAAAATGATGAGGAAATT |
| | | AGCATTACCAATCCGGACAGCATTGCATTTATGCAATCCTTATTAAC |
| | | AGAGGTAATTCATACCTTTGGCGATAGCACCAAGCATTTTCATATTG |
| | | GCGGAGATGAGTTTGGTTATGATGAAAATAGTAATCATGAATTTATT |
| | | ACCTATGCCAATAAATTGGCTGATTTTTTAAGAGAAAAAGGATTAAA |
| | | AACTCGAATTTGGAATGATGGTTTAATTAAAAATACCATAGATCAAT |
| | | TAAATCCTAATATTGAAATTACCTACTGGAGTTACGACGGC |
| 9 | Actinobacillus | ATGAAAAAAGCAATTACTTTATTTACATTATTATGCGCGGTACTTCTCTC |
| | pleuropneumoniae | ATTCAGTACCGCAACTTATGCAAACGCTATGGACTTACCTAAAAAAGAAA |
| | strain IA5 | GCGGTCTGACGTTAGATATCGCACGTCGTTTCTATACCGTTGATACGATA |
| | | AAACAATTTATCGATACGATTCATCAGGCGGGCGGCACTTTTCTGCATTT |
| | | ACATTTTTCCGATCACGAGAATTATGCATTGGAAAGTTCTTATTTGGAAC |
| | | AACGAGAAGAAAATGCGACCGAGAAAAACGGAACCTATTTCAATCCGAAA |
| | | ACAAATAAGCCGTTTCTCACTTATAAACAGCTCAATGAAATTATCTATTA |
| | | TGCCAAAGAACGAAATATTGAAATTGTGCCTGAAGTCGATAGCCCGAATC |
| | | ATATGACGGCGATTTTTGATCTTTTAACCCTTAAGCACGGAAAGGAATAC |
| | | GTAAAAGGGCTAAAATCGCCTTATATCGCCGAGGAAATCGATATTAATAA |
| | | CCCCGAAGCGGTTGAAGTTATTAAAACCTTAATCGGTGAAGTGATCTATA |
| | | TTTTCGGACATTCAAGCCGGCATTTCCATATCGGCGGAGATGAATTTAGC |
| | | TATGCGGTCGAAAATAATCATGAATTTATTCGGTATGTGAATACCTTAAA |
| | | TGATTTTATCAATTCCAAAGGGCTAATTACCCGTGTTTGGAATGACGGTT |
| | | TGATCAAAAACAACTTAAGCGAACTCAATAAAAACATTGAAATCACTTAC |
| | | TGGAGCTACGACGGTGACGCTCAAGCCAAAGAAGATATTCAATATCGACG |
| | | TGAAATAAGAGCCGATTTGCCCGAACTGCTGGCAAACGGTTTTAAGGTTT |
| | | TAAACTATAATTCTTATTATTTATACTTTGTGCCTAAATCCGGTTCTAAT |
| | | ATTCACAATGACGGTAAATATGCGGCTGAAGACGTATTAAATAACTGGAC |
| | | ATTAGGTAAATGGGACGGAAAAAACAGCTCAAATCACGTACAAAATACGC |
| | | AGAATATTATCGGTTCTTCTTTGTCGATTTGGGGGGAACGTTCCAGCGCA |
| | | TTAAATGAACAAACTATTCAGCAAGCCTCTAAAAATTTATTAAAAGCGGT |
| | | GATCCAAAAAACTAATGATCCGAAATCGCATTAG |
| | | (Accession No. AY618481.1 GI: 48727580) |
| 11 | Aggregatibacter | GTGTTTTATTGCATTCTTTCCTTTTTTGAGGCTTTTATGAAAAATATAAAAA |
| | aphrophilus ATCC | AAACTATCTTTTTACTTCCATCTTTGCTTTTGTTGAACCCGTTAAATAGTCT |
| | 33389 | TGCCGATGTAAAGATGTCTTCTGATAATTCCAATATAAATTCGCCAACAGTG |
| | | CAAAAAACAACTTTAAAGGAAAGCGGATTAATGCTGGATATTGCAAGGCACT |
| | | TTTATCCGCCTGAAGTTATTAAATCTTTTATTGATACTATTAGCCAGTCGGG |
| | | CGGGACATTTCTGCATTTACATTTTTCCGATCACGAAAATTATGCTTTAGAA |
| | | AGTCGGTTGCTGAATCAGCGTGTGGAAAATGCGATTCAAGATAAAAGCGGAA |
| | | TTTATATTAATCCTTACACCAATAAACCCTTCTTGAGCTATCAACAGCTTGA |
| | | TGACATTAAAGCATATGCAAAATCAAAAGGCATTGAGCTTATTCCCGAACTG |
| | | GATAGCCCGAATCACATGACGGCGATTTTTACGTTATTAAAACAAGAAAAGG |
| | | GGGAAGCCTATCTTCAATCTTTAAAATCACCACAAAATGATGACGAAATTAG |
| | | CATTACCAATCCGGACAGTATTGAGTTTATGCAATCTTTGTTGGCTGAAGTT |
| | | ATTCATATTTTTGGTGACAGTACTAAGCATTTTCATATTGGTGGTGATGAGT |
| | | TTGGTTATGATGAAAATAGTAATCATGAATTTATTGCCTATGCCAATAAATT |
| | | AGCTGATTTTTTAAGAGAAAAAGGATTAAAAACCCGAATTTGGAATGATGGT |
| | | TTAATTAAAAATACCGTCGATCAATTAAATCCTAATATTGAAATTACCTACT |
| | | GGAGTTATGATGGTGATACGGAAGATGAAGGCGAGGCAAATCGACGTCGTCA |
| | | TCTGCGGGTGAGTCTGCCGGAATTAGTCGAAAAAGGTTTTACTGTTTTAAAT |
| | | TATAATTCTTATTATCTTTATATAAATCCTAAAACCTCTTCAACGCTTTCGC |
| | | ATGATGCGAGCTTTGCTCAAAAAGACGTCTTAAATCATTGGGATCTTAGTGT |
| | | TTGGGATGGACAAAATACGGAAAATAAAGCACAAGATACCCATAAAATTGCC |
| | | GGCGCGGCTTTGTCTATTTGGGGAGAAGATGTCAAGGCCTTGAAAAGTGAAG |
| | | CCATTCAGAAAAATACAAAAGGCTTACTGGAGGCGGTGATTCAAAAGACGAA |
| | | CAGTGAGATTAAATAA |
| | | (Accession No. AEWB01000014.1 GI: 347814697) |
| 13 | Aggregatibacter | ATGCTGGATATTGCAAGGCACTTTTATCCGCCTGAAGTTATTAAATCTTTTA |
| | aphrophilus | TTGATACTATTAGCCAGTCGGGCGGAACATTTCTGCATTTACATTTTTCCGA |
| | strain F0387 | TCACGAAAATTATGCTTTAGAAAGCAGGTTGTTGAATCAGCGGGCAGAAAAC |
| | | GCAATTTTAAATAAAAACGGAATTTATATTAATCCTTACACCAATAAGCCTT |
| | | TCTTGAGTTATCAACAGTTGGATGACATTAAAGCATATGCAAAATTAAAAGG |
| | | TGTTGAGCTTATTCCCGAATTAGATAGCCCGAATCACATGACAGCGATTTTT |
| | | ACCTTATTAAAAAAAGAAAAAGGAAAAAATTATCTTCAATCGTTAAAATCAC |
| | | CACAAAATGATGAGGAAATTAGCATTACCAATCCGGACAGCATTGCATTTAT |
| | | GCAATCCTTATTAACTGAGGTAATTCATACCTTTGGCGATAGCACCAAGCAT |
| | | TTTCATATTGGCGGAGATGAGTTTGGTTATGATGAAAATAGTAATCATGAAT |
| | | TTATTGCCTATGCCAATAAATTAGCTGATTTTTTAAGAGAAAAAGGATTAAA |
| | | AACCCGAATTTGGAATGATGGTTTAATTAAAAATACCGTCGATCAATTAAAT |
| | | CCTAATATTGAAATTACCTACTGGAGTTATGACGGTGATACGGAAGATGAAG |
| | | GCGAGGCAAATAGACGTCGTCATCTTCGGGTGAGTTTGCCGGAATTGGTCGA |
| | | AAAAGGTTTTACTGTTTTAAATTATAATTCTTATTATCTTTATATAAATCCT |
| | | AAAACCTCTTCAACGCTTTCTCATGATGCGAGCTTTGCTCAAAAAGACGTCT |
| | | TAAATCATTGGGATCTTAGTGTTTGGGATGGACAAAATATGGAAAATAAAGC |
| | | ACAAGATACCCATAAAATTGCCGGCGCGGCTTTGTCTATTTGGGGGGAAGAT |
| | | GTCAAGGCCTTGAAAAGTGAAGCCATTCAGAAAAATACAAAAGGCTTACTGG |
| | | AGGCGGTGATTAAAAAGACAAATAGTGAGATTAAATAA |
| | | (Accession No. ACZJ01000017.1 GI: 353344797) |

TABLE 2
DspB Polypeptide Sequences

| SEQ | | |
|---|---|---|
| ID | | |
| NO: | Origin | Sequence |
| 2 | Aggregatibacter | MNYIKKIILSLFLLGLFSVLNCCVKGNSIYPQKTSTKQTG |
| | actinomycetemcomitans | LMLDIARHFYSPEVIKSFIDTISLSGGNFLHLHFSDHENY |
| | strain CU1000N | AIESHLLNQRAENAVQGKDGIYINPYTGKPFLSYRQLDDI |
| | | KAYAKAKGIELIPELDSPNHMTAIFKLVQKDRGVKYLQGL |
| | | KSRQVDDEIDITNADSITFMQSLMSEVIDIFGDTSQHFHI |
| | | GGDEFGYSVESNHEFITYANKLSYFLEKKGLKTRMWNDGL |
| | | IKNTFEQINPNIEITYWSYDGDTQDKNEAAERRDMRVSLP |
| | | ELLAKGFTVLNYNSYYLYIVPKASPTFSQDAAFAAKDVIK |
| | | NWDLGVWDGRNTKNRVQNTHEIAGAALSIWGEDAKALKDE |
| | | TIQKNTKSLLEAVIHKTNGDE |
| | | (Accession No. AY228551.1 GI: 30420959) |
| 4 | Actinobacillus ligniersii | DHENYALESSYLEQREENAVEKNGTYFNPKTNKPFLTYKQLNEIIYYA |
| | strain 19393 | KERNIEIVPEVDSPNHMTAIFDLLTLKHGKEYVKGLKSPYLAEEIDIN |
| | | NPEAVEIIKTLIGEVIYIFGHSSRHFHIGGDEFSYAVENNHEFIRYVN |
| | | TLNDFINNKGLITRIWNDGLIKNNLNELNRNIEITYWSYDG |
| 6 | Aggregatibacter | DHENYAIESHLLNQRAENAVQGKDGIYINPYTGKPFLSYRQLDDIKAY |
| | actinomycetemcomitans | AKAKGIELIPELDSPNHMTAIFKLVQKDRGIKYLQGLKSRQVDDEIDI |
| | strain IDH781 | TNADSIAFMQSLMSEVIDIFGDTSQHFHIGGDEFGYSVESNHEFITYA |
| | | NKLSYFLEKKGLKTRMWNDGLIKSTFEQINPNIEITYWSYDG |
| 8 | Aggregatibacter | DHENYALESRLLNQRAENAILNKNGIYINPYTNKPFLSYQQLDDIKAY |
| | aphrophilus | AKLKGIELIPELDSPNHMTAIFTLLKKEKGKNYLQSLKSPQNDEEISI |
| | strain NJ8700 | TNPDSIAFMQSLLTEVIHTFGDSTKHFHIGGDEFGYDENSNHEFITYA |
| | | NKLADFLREKGLKTRIWNDGLIKNTIDQLNPNIEITYWSYDG |
| 10 | Actinobacillus | MKKAITLFTLLCAVLLSFSTATYANAMDLPKKESGLTLDI |
| | pleuropneumoniae | ARRFYTVDTIKQFIDTIHQAGGTFLHLHFSDHENYALESS |
| | | YLEQREENATEKNGTYFNPKTNKPFLTYKQLNEIIYYAKE |
| | | RNIEIVPEVDSPNHMTAIFDLLTLKHGKEYVKGLKSPYIA |
| | | EEIDINNPEAVEVIKTLIGEVIYIFGHSSRHFHIGGDEFS |
| | | YAVENNHEFIRYVNTLNDFINSKGLITRVWNDGLIKNNLS |
| | | ELNKNIEITYWSYDGDAQAKEDIQYRREIRADLPELLANG |
| | | FKVLNYNSYYLYFVPKSGSNIHNDGKYAAEDVLNNWTLGK |
| | | WDGKNSSNHVQNTQNIIGSSLSIWGERSSALNEQTIQQAS |
| | | KNLLKAVIQKTNDPKSH |
| | | (Accession No. AAT46094.1 GI: 48727581) |
| 12 | Aggregatibacter | MFYCILSFFEAFMKNIKKTIFLLPSLLLLNPLNSLADVKMSSDNSNIN |
| | aphrophilus ATCC | SPTVQKTTLKESGLMLDIARHFYPPEVIKSFIDTISQSGGTFLHLHFS |
| | 33389 | DHENYALESRLLNQRVENAIQDKSGIYINPYTNKPFLSYQQLDDIKAY |
| | | AKSKGIELIPELDSPNHMTAIFTLLKQEKGEAYLQSLKSPQNDDEISI |
| | | TNPDSIEFMQSLLAEVIHIFGDSTKHFHIGGDEFGYDENSNHEFIAYA |
| | | NKLADFLREKGLKTRIWNDGLIKNTVDQLNPNIEITYWSYDGDTEDEG |
| | | EANRRRHLRVSLPELVEKGFTVLNYNSYYLYINPKTSSTLSHDASFAQ |
| | | KDVLNHWDLSVWDGQNTENKAQDTHKIAGAALSIWGEDVKALKSEAIQ |
| | | KNTKGLLEAVIQKTNSEIK |
| | | (Accession No. EGY31409.1 GI: 347814761) |
| 14 | Aggregatibacter | MLDIARHFYPPEVIKSFIDTISQSGGTFLHLHFSDHENYALESRLLNQRAENA |
| | aphrophilus | ILNKNGIYINPYTNKPFLSYQQLDDIKAYAKLKGVELIPELDSPNHMTAIFTL |
| | strain F0387 | LKKEKGKNYLQSLKSPQNDEEISITNPDSIAFMQSLLTEVIHTFGDSTKHFHI |
| | | GGDEFGYDENSNHEFIAYANKLADFLREKGLKTRIWNDGLIKNTVDQLNPN |
| | | IEITYWSYDGDTEDEGEANRRRHLRVSLPELVEKGFTVLNYNSYYLYINPKTS |
| | | STLSHDASFAQKDVLNHWDLSVWDGQNMENKAQDTHKIAGAALSIWGEDVKAL |
| | | KSEAIQKNTKGLLEAVIKKTNSEIK |
| | | (Accession No. EHB89189.1 GI: 353344889) |

Expression of DispersinB polypeptides

Polynucleotides encoding DispersinB polypeptides can be prepared by known recombinant methods. These methods include, but are not limited to, isolation from a natural source, PCR, and the like. A polynucleotide encoding a DispersinB polypeptide may be expressed directly or as a fusion with another polypeptide in a plant, such as a crop.

Expressing DispersinB in plants utilizes standard recombinant procedures to produce DispersinB polypeptides. The expression of a plant polynucleotide that exists as double-stranded DNA involves the transcription of one strand of the DNA by RNA polymerase to produce messenger RNA (mRNA), and processing of the mRNA primary transcript inside the nucleus. This processing involves a 3′ non-translated region which adds polyadenylate nucleotides to the 3′ end of the mRNA. A 3′ non-translated region contains a polyadenylation signal which functions in plants to cause the addition of polyadenylate nucleotides to the 3′ end of the RNA. Examples of suitable 3′ regions are (1) the 3′ transcribed, non-translated regions containing the polyadenylate signal of the tumor inducing (Ti) plasmid genes of Agrobacterium such as the nopaline synthase (NOS) gene, and (2) plant genes like the soybean storage protein gene and the ssRUBISCO.

A heterologous polynucleotide encoding a DispersinB polypeptide is inserted into a replicable vector for expression in the plant. Many vectors are available for this purpose, and selection of the appropriate vector will depend mainly on desired expression levels and the particular host cell'to be transformed with the vector. Each vector contains various components depending on its function (amplification of DNA or expression of DNA) and the particular host cell with which it is compatible. The vector components for plant transformation generally include, but are not limited to, one or more of the following: origin of replication, one or more marker gene, and inducible promoter. Examples of suitable vectors are described herein. In an embodiment of the invention, vectors can contain a promoter under high regulation operably linked to a polynucleotide encoding a DispersinB polypeptide. Examples of suitable promoters are described herein, and include the promoters, and other such promoters under tight control, for example, by both positive and negative control elements.

There are a number of promoters that are active in plant cells. Such promoters include, but are not limited to, nonpaline synthase (NOS), octopine synthase (OCS), and mannopine synthase (MAS) promoters which are carried on tumor-inducing plasmids of Agrobacterium tumefaciens, the cauliflower mosaic virus (CaMV) 19S and 35S promoters, ribulose bis-phosphate carboxylase (ssRUBISCO, a very abundant plant polypeptide). Suitable promoters may include both those which are derived from a gene which is naturally expressed in plants and synthetic promoter sequences which may include redundant or heterologous enhancer sequences. A promoter selected should be capable of causing sufficient expression to result in the production of an effective amount of DispersinB enzyme to inhibit formation and growth of bacterial biofilms. The amount of DispersinB enzyme needed to inhibit and disperse biofilms may vary with the particular bacterial plant pathogen(s). Accordingly, the CaMV 35S, ssRUBISCO, and MA-S promoters are preferred. Further, a promoter may be tissue specific, such as a fruit specific promoter (e.g., 2A11 (Van Haaren et al., 1993); 2A12 (Zichao et al., 2002); and E-8 (Krasnyanski et al., 2001)) or a root specific promoter (e.g., PHT1 (Koyama et al., 2005); RB7 (Yammamoto et al., 1991); and FaRB7 (Vaughan et al., 2006).

In general, plasmid vectors containing replicon and control sequences derived from species compatible with the plant host cell are used. The vector ordinarily carries a replication site, as well as marking sequences that are capable of providing phenotypic selection in transformed cells.

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of plants.

Expression and cloning vectors also generally contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media. Those cells that are successfully transformed with a heterologous gene produce a protein conferring drug resistance and thus survive the selection regimen.

Transcription of DNA to produce mRNA is regulated by a promoter. The promoter region contains a sequence of nucleotides which signals RNA polymerase to associate with the DNA, and initiate the production of mRNA transcript using the DNA strand downstream from the promoter as a template to make a corresponding strand of RNA. Promoters can be induced utilizing standard methods. See, for example, Sambrook et al., Molecular Cloning: A Laboratory Manual (New York: Cold Spring Harbor Laboratory Press, 1989). In an embodiment, a DispersinB polynucleotide can be operably linked to a promoter that regulates transcription in a plant. The promoter can be any promoter that can regulate transcription in a plant.

mRNA produced by the dspB gene also contains a 5′ non-translated leader sequence. This sequence may be derived from the particular promoter selected such as the CaMV 35S, ssRUBISCO or M-AS promoters. The 5′ non-translated region may also be obtained from other suitable eukaryotic genes or a synthetic gene sequence. The requisite functionality of the 5′ non-translated leader sequence is to enhance translation of the mRNA in production of the encoded protein.

A dspB polynucleotide also contains a structural coding sequence that encodes a DispersinB enzyme or an active fragment thereof. Accordingly, a structural coding sequence from A. actinomycetemcomitans and active fragments thereof are disclosed. A dspB polynucleotide can be inserted into the genome of a plant by any suitable method. Suitable plant transformation vectors include those derived from a Ti plasmid of A. tumefaciens. In addition to plant transformation vectors derived from the Ti or root-inducing (Ri) plasmids of Agrobacterium, alternative methods can be used to insert a dspB polynucleotide of this invention into plant cells. A dspB polynucleotide could be first cloned into pENTR TOPO vector and then into Gateway destination vectors, which can subsequently be transformed into A. tumefaciens cells by any method (Herrera-Estralla, et al. 1983, Nature 303:209; Bevan, et al. 1983, Nature 304: 184; Klee, et al. 1985, Bio/Technology 3:637; Fraley, et al. 1985, Bio/Technology 3: 629). Such methods may involve, but are not limited to, liposomes, electroporation, and chemicals which increase free uptake of DNA, and the use of viruses or pollen as vectors. If desired, more than one polynucleotide may be inserted into the chromosomes of a plant, by methods such as repeating the transformation and selection cycle more than once. To generate a transgenic plant, tobacco leaf discs could be co-cultured with A. tumefaciens carrying a dspB construct.

Gene expression can be measured in a sample indirectly, for example, by conventional northern blotting to quantitate the transcription of mRNA (Thomas, 1980, Proc. Natl. Acad. Sci. USA, 77: 5201-5205). Various labels may be employed, most commonly radioisotopes, particularly ³²P. Other techniques may also be employed, such as biotin labeling. Biotin-modified nucleotides introduced into a polynucleotide can serve as the site for binding to avidin or antibodies that can be labeled with a wide variety of labels, such as radionucleotides, fluorescers, enzymes, or the like. Gene expression can also be measured directly, by analysis of expressed polypeptides, for example by western blot. If no dspB mRNA is detected, a promoter used in a chimeric polynucleotide construct is replaced with another, potentially stronger promoter and the altered construct retested. Alternatively, levels of DispersinB polypeptide may be assayed by immunoassay such as a Western blot. In many cases the most sensitive assay for DispersinB is a biofilm assay. Furthermore, the ability of a transformed plant to resist E. carotovora or R. solanacearum infection, biofilm formation, or biofilm growth can also be tested to confirm expression of a DispersinB enzyme.

Monitoring can also be affected in whole regenerated plants. When adequate production of dspB is achieved, and transformed cells have been regenerated into whole plants, the latter are screened for resistance to bacterial biofilm infection. Choice of methodology for the regeneration step is not critical, with suitable protocols being available for hosts from Solanaceae (potato, tomato, peppers, egg plants, etc.), Cruciferae (cabbage, radish, rapeseed, etc.), Rutaceae (orange, lemon, grape fruit, etc.), Fabaceae (pea nut or ground nut), Euphorbiaceae (cassava), Convolvulaceae (sweet potato), Umbelliftrae (carrot, celery, parsnip, etc.), Cucurbitaceae (melons, cucumbers, etc.), Leguminosae (alfalfa, soybean, beans, etc.), Graminaceae (wheat, rice, corn, etc.), and various floral as well as ornamental plants.

Examples of plants that can express DispersinB include, but are not limited to, corn, wheat, rice, millet, oat, barley, sorghum, sunflower, sweet potato, alfalfa, sugar beet, Brassica species, tomato, pepper, soybean, tobacco, melon, squash, potato, peanut, pea, cotton, citrus (e.g., orange, grapefruit, etc.), and cacao.

### Methods of Transforming Plants

“Introducing” a nucleotide construct into a plant refers to inserting the nucleotide construct into a plant cell. Methods for introducing nucleotide constructs into plants are well known and include, but are not limited to, stable transformation methods, transient transformation methods, and virus-mediated methods.

Plant transformation methods include transferring heterologous DNA into target plant cells, followed by applying selective pressure to recover transformed plant cells from a group of untransformed cell mass. Descriptions of methods for generating transgenic plants can be found in Ayres and Park, Crit. Rev. Plant Sci. 13:219-239 (1994) and Bommineni & Jauhar, Maydica 42:107-120 (1997).

Generation of transgenic plants may be performed by one of several methods, including, but not limited to, introduction of heterologous DNA by Agrobacterium into plant cells (Agrobacterium-mediated transformation), bombardment of plant cells with heterologous foreign DNA adhered to particles, and various other non-particle direct-mediated methods to transfer DNA (e.g., Hiei et al., Plant J. 6:271-282 (1994); Ishida et al., Nat. Biotechnol. 14:745-750 (1996); Ayres and Park 1994; and Bommineni & Jauhar, 1997).

### Methods of Producing DispersinB in Transgenic Plants

A polynucleotide that functions in plants and produces transgenic plants that exhibit antibiofilm activity against bacterial plant pathogens is described. A DispersinB polypeptide can be extracted from the transgenic plants by known methods.

A method for transforming plants to express a DispersinB polypeptide is disclosed. A transgenic plant expressing DispersinB can be resistant to biofilm formation. A transgenic plant expressing DispersinB can inhibit biofilm formation and/or biofilm growth. A method is described of harvesting and purifying DispersinB expressed in the plant. DispersinB can be part of a plant extract or purified to be substantially pure. Substantially pure DispersinB can be at least 90%, 95%, 96%, 97%, 98%, 99%, or 100% free of other plant materials. Another embodiment includes expressing a fusion protein, wherein the fusion protein is a DispersinB enzyme linked to a second polypeptide. The second polypeptide can be an antimicrobial polypeptide.

A transgenic plant expressing DispersinB at a level that is effective in inhibiting biofilm formation on plants and disrupting preformed biofilms of pathogens on plants include corn, wheat, rice, millet, oat, barley, sorghum, sunflower, sweet potato, alfalfa, sugar beet, Brassica species, tomato, pepper, soybean, tobacco, melon, squash, potato, peanut, pea, cotton, citrus (e.g., orange, grapefruit, etc.), and cacao. Such expression can inhibit plant pathogens such as Ralstonia solanacearum, Xanthomonas axonopodis, or Psuedomonas fluorsescens.

### Compositions

A composition includes a plant extract of DispersinB. The extract can be a crude extract or a substantially pure extract less than 100% pure. A composition can include a plant extract of DispersinB in combination with at least one antimicrobial, wherein the extract can be a crude extract or a substantially pure extract less than 100% pure. Also, antimicrobials including, but not limited to, rifampicin, cefamendole nafate, nitrofurazone, ciprofloxacin, silver compounds, bismuth thiols, ovotransferrin, lactoferrin, cationic antimicrobial peptides (CAP), bismuth ethanedithiol (BisEDT), nitrofurazone, sodium usnate, and mixtures thereof can be used in combination with DspB. The antimicrobial can also be an antibiotic.

### EXAMPLES

### Example 1

### Construction of Binary Vector

The DispersinB coding region was amplified from pRC3 (Ramasubbu et al., 2005) by PCR using Pfu Turbo DNA polymerase (Stratagene). The forward primer (5′-CACCATGAATTGTTGCG TAAAAGGCAATTC-3′; SEQ ID NO: 15) was used to create a CACC (SEQ ID NO:16) site (bold) adjacent to the DispersinB initiation codon in the open reading frame, and the reverse primer (5′-TTAGTGGTGGTGGTGGTG GTGCTCATCCCCATTCGTCTTATG-3′; SEQ ID NO:17) was designed to include the His-tag and stop codon. The PCR product was:

(SEQ ID NO: 18)
ATGAATTGTTGCGTAAAAGGCAATTCCATATATCCGCAAAAAACAAGTA
CCAAGCAGACCGGATTAATGCTGGACATCGCCCGACATTTTTATTCACC
CGAGGTGATTAAATCCTTTATTGATACCATCAGCCTTTCCGGCGGTAAT
TTTCTGCACCTGCATTTTTCCGACCATGAAAACTATGCGATAGAAAGCC
ATTTACTTAATCAACGTGCGGAAAATGCCGTGCAGGGCAAAGACGGTAT
TTATATTAATCCTTATACCGGAAAGCCATTCTTGAGTTATCGGCAACTT
GACGATATCAAAGCCTATGCTAAGGCAAAAGGCATTGAGTTGATTCCCG
AACTTGACAGCCCGAATCACATGACGGCGATCTTTAAACTGGTGCAAAA
AGACAGAGGGGTCAAGTACCTTCAAGGATTAAAATCACGCCAGGTAGAT
GATGAAATTGATATTACTAATGCTGACAGTATTACTTTTATGCAATCTT
TAATGAGTGAGGTTATTGATATTTTTGGCGACACGAGTCAGCATTTTCA
TATTGGTGGCGATGAATTTGGTTATTCTGTGGAAAGTAATCATGAGTTT
ATTACGTATGCCAATAAACTATCCTACTTTTTAGAGAAAAAAGGGTTGA
AAACCCGAATGTGGAATGACGGATTAATTAAAAATACTTTTGAGCAAAT
CAACCCGAATATTGAAATTACTTATTGGAGCTATGATGGCGATACGCAG
GACAAAAATGAAGCTGCCGAGCGCCGTGATATGCGGGTCAGTTTGCCGG
AGTTGCTGGCGAAAGGCTTTACTGTCCTGAACTATAATTCCTATTATCT
TTACATTGTTCCGAAAGCTTCACCAACCTTCTCGCAAGATGCCGCCTTT
GCCGCCAAAGATGTTATAAAAAATTGGGATCTTGGTGTTTGGGATGGAC
GAAACACCAAAAACCGCGTACAAAATACTCATGAAATAGCCGGCGCAGC
ATTATCGATCTGGGGAGAAGATGCAAAAGCGCTGAAAGACGAAACAATT
CAGAAAAACACGAAAAGTTTATTGGAAGCGGTGATTCATAAGACGAATG
GGGATGAGCACCACCACCACCACCACTAA;

which encodes

(SEQ ID NO: 19)
MNCCVKGNSIYPQKTSTKQTGLMLDIARHFYSPEVIKSFIDTISLSGGN
FLHLHFSDHENYAIESHLLNQRAENAVQGKDGIYINPYTGKPFLSYRQL
DDIKAYAKAKGIELIPELDSPNHMTAIFKLVQKDRGVKYLQGLKSRQVD
DEIDITNADSITFMQSLMSEVIDIFGDTSQHFHIGGDEFGYSVESNHEF
ITYANKLSYFLEKKGLKTRMWNDGLIKNTFEQINPNIEITYWSYDGDTQ
DKNEAAERRDMRVSLPELLAKGFTVLNYNSYYLYIVPKASPTFSQDAAF
AAKDVIKNWDLGVWDGRNTKNRVQNTHEIAGAALSIWGEDAKALKDETI
QKNTKSLLEAVIHKTNGDEHHHHHH

The PCR product was directionally cloned into the pENTR TOPO® vector (Invitrogen). The DispersinB construct was sequenced to confirm that the coding region was in frame. LR clonase reactions to transfer DNA fragments from entry clones to Gateway destination vectors were carried out. The pMDC32 Gateway destination vector (ABRC Stock Center at Ohio State University) was used for the expression of DispersinB protein.

### Example 2

### Plant Transformation

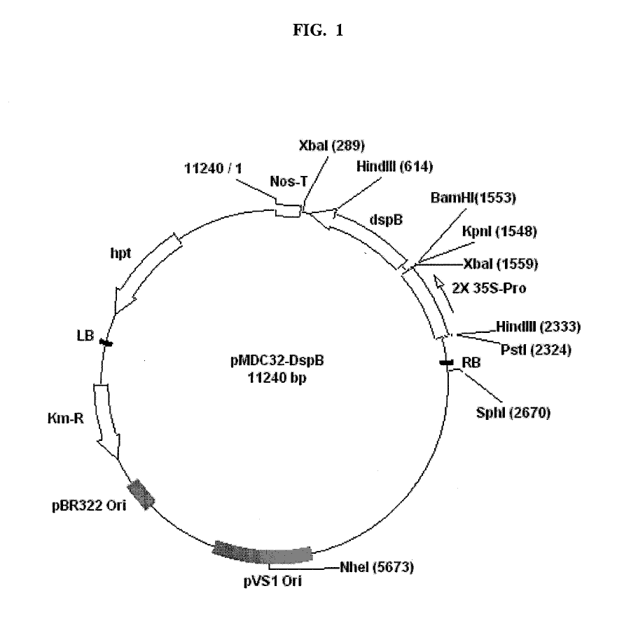

The pMDC32-DspB construct (FIG. 1; ATCC accession no. ______), which contains the dspB gene from A. actinomycetemcomitans operably linked to the Cauliflower mosaic virus promoter 2×35S, was transformed into Agrobacterium tumefaciens LBA4404 cells (Invitrogen) by electroporation, and selected on YM plates containing 50 μg/mL kanamycin. The Tobacco seeds (Nicotiana tabacum cv. Havana 38) were obtained from Lehle Seeds (Texas, USA). The healthy fully expanded leaves from 4-5 week old tissue culture grown tobacco plants were used for transformation. Leaf discs were co-cultured with A. tumefaciens LBA4404 carrying pMDC32-DspB over a period of 48 h in MS medium. Explants were sub-cultured in regeneration medium (MS salts and vitamins, 20 g/L sucrose, 2.5 g/L phytagel, pH 5.8, plus 50 mg/mL hygromycin and 250 mg/mL cefotaxime) and the callus was transferred to fresh medium until distinct shoots appeared. Shoots were grown in micro-propagation medium (MS salts, 20 g/L sucrose, 2.5 g/L phytagel, pH 5.8) supplemented with hygromycin and cefotaxime. Plants from different transgenic lines were maintained under standard conditions (16 h light/8 h dark cycle at 20° C. to 22° C.).

### Example 3

### Staphylococcus epidermidis Biofilm Dispersal Activity of Leaf Extract

For detachment assay, leaf extracts were prepared from fresh tobacco leaf tissue (500 mg) of different transgenic plants. Briefly, leaf tissues were ground in liquid nitrogen and immediately transferred to 1.5 mL of protein extraction buffer (20 mM HEPES pH 7.4, 1 mM MgSO4, 1 mM CaCl₂ and 1% Triton-X® 100). After vortexing for 30 seconds, samples were centrifuged at 14,000 g for 10 min at 4° C. The supernatant was collected and glycerol was added to a final concentration of 10% and kept at −80° C. S. epidermidis biofilm was prepared by following the method of (Kaplan et al., 2004, Antimicrob Agents Chemother., 48:2633-6.). Briefly, two loops of colonies scraped from the surface of an agar plate were transferred to a microfuge tube containing 200 μL fresh medium. Cells were homogenized with a disposable pellet Kontes pestle (Fischer, Itasca, Ill.) and vortex agitated at high speed for 30 s. Twenty-two microliters were transferred to a 50 mL polystyrene tube containing 22 mL fresh medium. The resulting inoculum contained 10⁹-10¹⁰ cfu/mL. Biofilms were grown in 96-well tissue-culture-treated polystyrene microtiter plates (Corning model 3595, Sigma-Aldrich). Wells filled with 200 μL inoculum were incubated for 16 h at 37° C.

For detachment studies, wells were first rinsed by submerging the entire plate in a tub of cold, running tap water after which 50 μL of leaf extract was added to each well along with 150 μL of PBS buffer. Plates were incubated at 37° C. at different time periods. After removing the leaf extract, the biofilms were washed in tub of cold, running tap water and were stained with crystal violet as previously described (Kaplan and Fine, 2002, Appl Environ Microbiol., 68: 4943-50). The absorbance values of the well solutions were determined by using a Bio-Rad Benchmark® microplate reader set at 590 nm. All assays were performed in triplicate wells on at least three separate occasions and exhibited similar results with minimal variation among them.

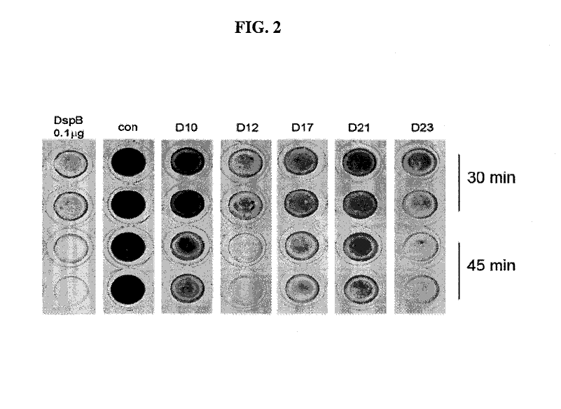

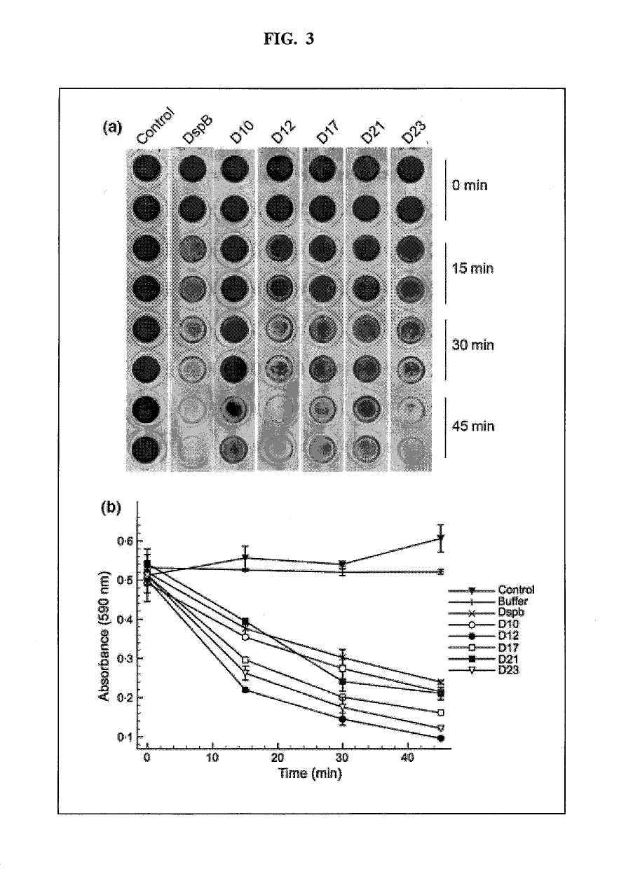

Extracts from transgenic tobacco plant expressing DispersinB dispersed S. epidermidis biofilm, whereas vector-transformed control plant extract did not disperse S. epidermidis biofilm (FIG. 2). The detachment assay using S. epidermidis showed that DspB-expressing transgenic tobacco plants produced substantial amounts of the biologically active enzyme although at varying levels. Leaf extract from transgenic plant D10 detached about 60% of biofilm after 45 min of incubation, whereas extract from vector-transformed control plants resulted in no detachment (FIG. 3a). Among the transgenic lines, extract from plant D12 showed the maximum detachment of biofilm (82%) (FIG. 3b) after 45 min.

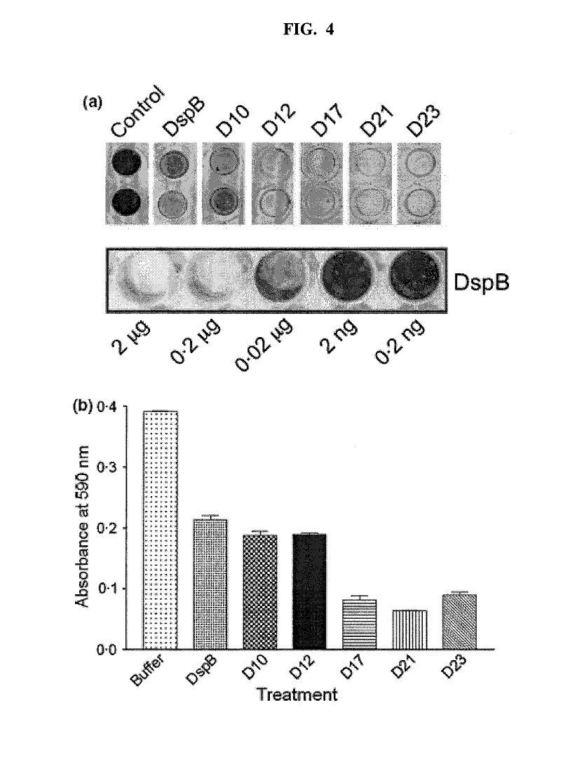

The extracts from the transgenic plants not only detached the preformed S. epidermidis biofilm but also inhibited its biofilm formation, suggesting that the recombinant enzyme in the leaves is biologically active (FIG. 4a). While most of the transgenic plants showed at least 70% inhibition of biofilm formation, line D10 exhibited only 33% inhibition (FIG. 4b). The biofilm detachment and inhibition collectively show that the expressed DspB in these plants is biologically active. The variation of DspB expression between different transgenic lines could be the result of differences in transgene copy number and/or position and length of the transgene integration event.

### Example 4

### Biofilm Inhibition Activity of Leaf Extract

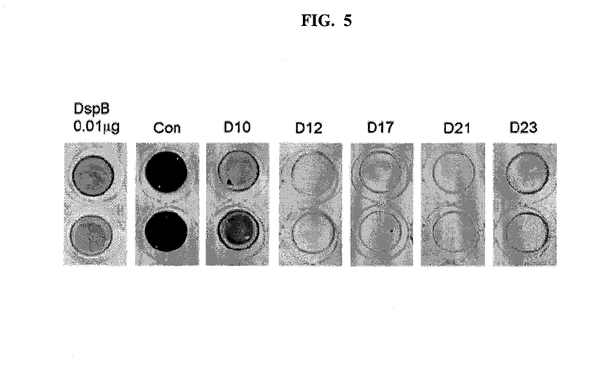

Twenty two microliters of S. epidermidis was transferred to 16.5 mL fresh medium and wells were filled with 150 μL of inoculum with 50 μL of leaf extracts from control and transgenic plants to obtain 10⁷ to 10⁸ CFU/mL. The plates were incubated for 20 h at 37° C. after which the plates were washed in cold running water and stained with crystal violet as described above. Plant extract from transgenic tobacco expressing DispersinB inhibited S. epidermidis biofilm formation, whereas vector-transformed control extracts did not inhibit S. epidermidis biofilm formation (FIG. 5).

### Example 5

### Western Blot Analysis of Leaf-Extracted DispersinB

Total protein was extracted from leaves using Plant total protein extraction kit (Sigma) according to the manufactures instructions. Protein concentration was estimated using Bio-Rad protein assay reagent. The 10 μg of total protein was resolved on SDS-PAGE and transferred to a nitrocellulose membrane. An immunoblot was performed as per standard protocol. Briefly, the membrane was blocked with 5% milk in TBS was incubated with purified polyclonal antibodies. After washing, the membrane was incubated with HRP-linked secondary antibodies. The membrane was developed with chemiluminescent substrate (Pierce, Thermo Scientific).

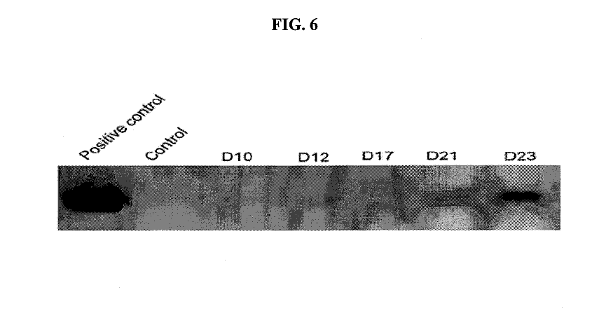

Among 33 transformants generated, 22 plants were positive for the expression of recombinant DspB protein as determined from the results of western blots (data not shown). From the 22 positive lines, five lines showing a range of DspB expression levels (lowest to highest levels) were selected for further studies. Among these five lines, the expression levels were in the order D10<D17<D12<D21<D23 (FIG. 6). All transformants were judged to be phenotypically normal as there was no morphological variation in growth and development compared to the wild type. A DispersinB protein band was not observed from vector-transformed control plant extract (FIG. 6). DspB was expressed in the cytosol and was not surface bound nor secreted.

### Example 6

### Extraction and Detection of Polysaccharide in Pcc

Polysaccharide was purified from the bacterium as described by Frank & Patel (Infect. Immun. 2007; 75: 4728-4742). Briefly, Pcc were grown overnight in Petri dishes containing LB medium at 28° C. Cells were harvested and washed twice in PBS, resuspended in 0.5 M EDTA, sonicated for 5 min at 40 kHz in a bath sonicator, boiled for 5 min, and centrifuged. Supernatant was treated with 200 μg proteinase K for 30 min at 65° C., and then the enzyme was heat-inactivated for 15 min at 80° C. Extracts were spotted on PVDF (Bio-Rad) membranes using dot-blot apparatus (Bio-Dot®, Bio-Rad), and the blots were blocked with 3% bovine serum albumin (BSA) in TBS overnight. The blots were first probed with goat anti-deacetylated PNAG antibody (1:5000) in TBS-0.1% Tween®-20 containing 3% BSA for 90 min at 4° C. After washing, the blots were further probed with HRP-linked rabbit anti-goat antibody (1:5000) in 0.1% TBS Tween®-20 containing 3% BSA for 90 min and developed with chemiluminescent substrate (as above). Several controls were tested in this assay, including DspB-treated polysaccharide, N-acetylglucosamine, a tetramer of β(1,6)linked N-acetylglucosamine and a tetramer of β(1,4)linked N-acetylglucosamine.

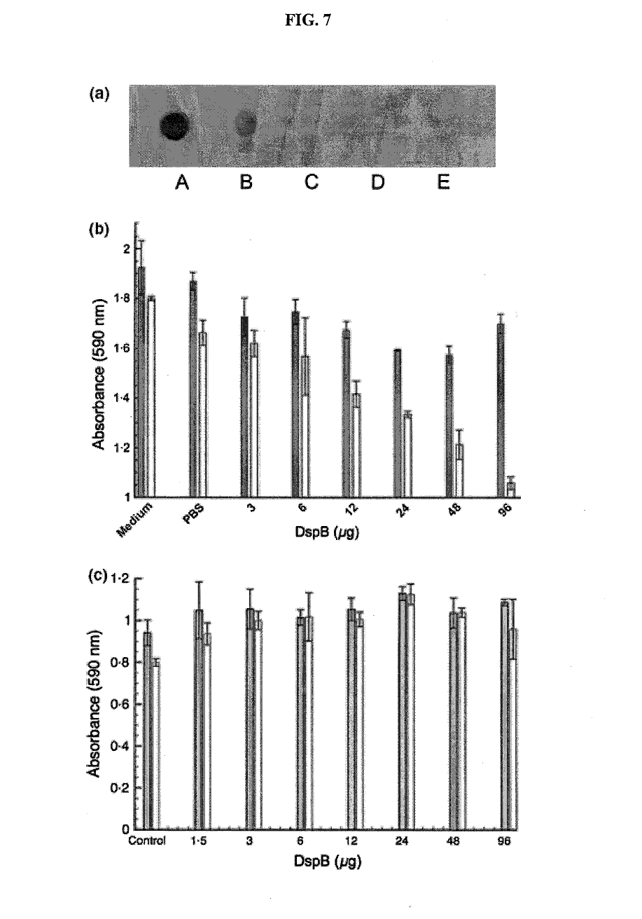

Exopolysaccharide analysis of P. carotovorum subsp. carotovorum (ATCC 15713) suggested that this bacterium also synthesizes a PNAG/PGA/PIA-like material (FIG. 7a). Therefore, it is likely that the individual proteins encoded by the loci might have similar functions that have been proposed for the corresponding proteins of the PGA-producing bacteria. More importantly, the presence of the pga locus renders the bacterium susceptible to biofilm detachment by the enzyme DspB.

### Example 7

### Detachment and Inhibition Assay for P. carotovorum subsp. Carotovorum

The question of whether DspB inhibits the formation of biofilm by Pcc was tested using an in vitro microtitre plate assay (Kaplan et al., Antimicrob. Agents Chemother. 2004; 48: 2633-2636).

P. carotovorum subsp. Carotovorum (ATCC 15713) cells were cultured overnight at 28° C. in LB. Pcc cells were centrifuged (5000 g for 10 min) and diluted to density of 1×10⁸ CFU mL⁻¹ with fresh medium. Biofilms were grown in 96-well tissue-culture-treated polystyrene microtiter plates. Wells were filled with 200 μL inoculum with different concentrations of native and boiled DspB and incubated for 24 h at 28° C. Biofilms were washed with cold running water and residual biofilms were stained with crystal violet as described above. The absorbance values of the well solutions were determined at 590 nm. For detachment studies, wells filled with inoculum (200 μL) were incubated for 24 h at 28° C. Wells were first rinsed with PBS (3×) and treated with different concentrations of native and boiled DspB for 1 h at 37° C. Biofilms were stained with crystal violet and absorbance measured at 590 nm.

As evident in FIG. 7b, DspB inhibited the Pcc biofilm formation in a dose-dependent manner. Interestingly, DspB did not detach Pcc biofilms in microtitre plates (FIG. 7c), reminiscent of the effect of DspB on S. aureus as well as A. actinomycetemcomitans biofilms. The biofilms of these bacteria are more complex and contain DNA as well as proteins.

### Example 9

### Inhibition Assay for Topical Application of DspB on Tobacco Leaves

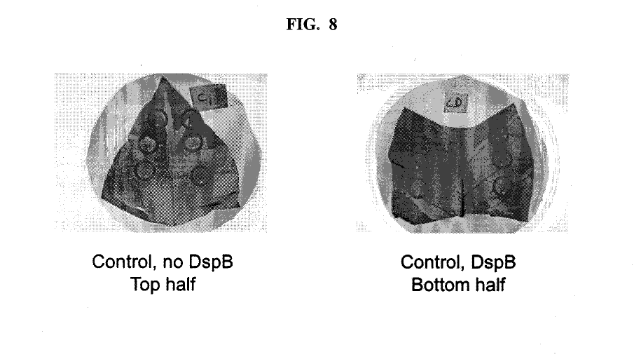

Protection of tobacco leaves by DspB from Pcc infection was tested. P. carotovora subsp. Carotovorum (Pcc) (ATCC 15713) cells were prepared by centrifugation (5000×g for 10 min) from an overnight culture and diluted to different population densities in fresh medium. A leaf was excised from the control plant (5 to 8 week old) and cut into two halves. These halves were placed in Petri dishes that contained 2 layers of Whatman filter papers moistened with sterile water. The leaf-halves were punctured slightly with 20 μL pipette tip (6 sites on each half) and each spot was treated with 10 μL of 1 mg/mL solution of DspB. The injured spots were then inoculated by adding 3 μL of Pcc. The inoculum cell numbers on the top, middle and lower two spots of each half were: 1×10³, 1×10⁴, and 1×10⁵ CFU, respectively. The photograph was taken 48 h after inoculation (FIG. 8).

Topical administration of DspB did not prevent or inhibit Pcc infection (FIG. 8). Tobacco leaves with DspB topically applied displayed tissue maceration similar to the tobacco leaves without topical application of DspB.

### Example 10

### Plant Leaf Infection Model of Transgenic DspB Plants

Since DspB was expressed in the cytosol and not on the surface, nor was it secreted, tests were undertaken to examine whether the in vitro results could be reproduced in vivo.

P. carotovorum subsp. carotovorum (Pcc) (ATCC 15713) cells were prepared by centrifugation (5000×g for 10 min) from an overnight culture and diluted to different population densities in fresh medium. Leaves excised from both control and transgenic lines of the same age (5 to 8 week old plants) were placed in Petri dishes that contained 2 layers of Whatman filter papers moistened with 4 mL of sterile water. 3 μL of Pcc from various diluted samples were added to the freshly wounded leaf surface. The leaves were punctured slightly with 20 μL pipette tip and were incubated at 28° C. for 24 and 48 h. Bacteria from the infected sited from the transgenic lines were reisolated by excision of the site, transferred to 50 sterile water, and vortexed. Five microlitres of this bacterial suspension were used for reinoculation of control leaves to test their viability and ability to infect new leaves. The leaves were then examined after 24 h of incubation.

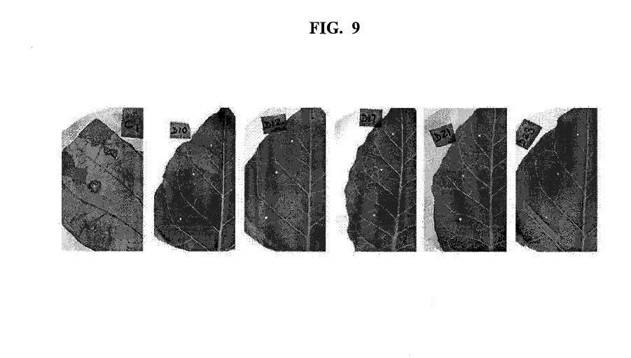

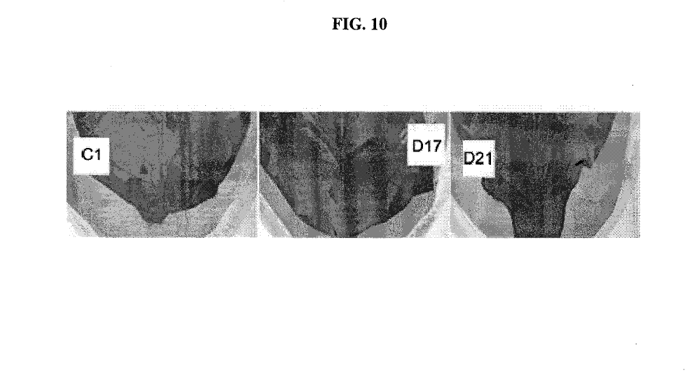

The five DispersinB transgenic lines showed no maceration even with a high density of inoculum (1×10⁵/3 μL) up to 48 h (FIG. 9). In contrast, control plants (wild type) displayed typical maceration symptoms within 24 h after inoculation (see, e.g., first panel of FIG. 9). Pcc were isolated from each of C1 wild type leaves and transgenic leaves D17 and D21 in FIG. 8. Wild type leaves were infected with resuspended Pcc from each of C1, D17, and D21. The bacteria isolated from the transgenic lines used to then infect wild type leaves caused tissue maceration within 24 hours (FIG. 10) similar to the maceration seen in the control wild type leaves. Thus, expression of DspB in transgenic tobacco plants protected the tobacco against local infections. Even the transgenic line D10, which produces lower levels of DspB expression, demonstrated resistance to Pcc infection.

1. A transgenic plant comprising a DispersinB polypeptide, variant, or fragment thereof.

2. The transgenic plant of claim 1, wherein the DispersinB polypeptide comprises at least 90% sequence identity to SEQ ID NO: 2, 4, 6, 8, 10, 12, or 14.

3. A transgenic plant comprising a DispersinB polynucleotide, variant, or fragment thereof.

4. The transgenic plant of claim 3, wherein the DispersinB polynucleotide comprises at least 90% sequence identity to SEQ ID NO: 1, 3, 5, 7, 9, 11, or 13.

5. (canceled)

6. (canceled)

7. (canceled)

8. (canceled)

9. (canceled)

10. The transgenic plant according to claim 1, wherein the transgenic plant is selected from the plant families consisting of Solanaceae, Cruciferae, Rutaceae, Fabaceae, Euphorbiaceae, Convolvulaceae, Umbelliferae, Cucurbitaceae, Leguminosae, and Graminaceae.

11. (canceled)

12. The transgenic plant according to claim 1, wherein the transgenic plant is selected from the group consisting of potato, tomato, peppers, egg plants, cabbage, radish, rapeseed, orange, lemon, grape fruit, pea nut or ground nut, cassava, sweet potato, carrot, celery, parsnip, melons, cucumbers, alfalfa, soybean, beans, wheat, rice, and corn.

13. A transgenic plant comprising a fusion protein of a DispersinB polypeptide linked to a second polypeptide.

14. The transgenic plant of claim 13, wherein the DispersinB polypeptide is at least 90% identical to SEQ ID NO: 2, 4, 6, 8, 10, 12, or 14.

15. A transgenic plant comprising a DispersinB polynucleotide linked to a second polynucleotide that encodes a fusion protein of a DispersinB polypeptide linked to a second polypeptide.

16. The transgenic plant of claim 15, wherein the DispersinB polynucleotide is SEQ ID NO: 1, 3, 5, 7, 9, 11, or 13.

17. The transgenic plant of claim 13, wherein the DispersinB polypeptide comprises at least 90% sequence identity to SEQ ID NO: 2, 4, 6, 8, 10, 12, or 14.

18. The transgenic plant of 13, wherein the DispersinB polypeptide is SEQ ID NO: 2, 4, 6, 8, 10, 12, or 14.

19. The transgenic plant of claim 15, wherein the DispersinB polynucleotide comprises at least 90% sequence identity to SEQ ID NO: 1, 3, 5, 7, 9, 11, or 13.

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. A transgenic plant seed comprising a DispersinB polynucleotide, variant, or fragment thereof.
